(19) United States
(12) Patent Application Publication
(10) Pub. No.: US 2018/0049758 A1
(43) Pub. Date: Feb. 22, 2018

(54) PATIENT-SPECIFIC SURGICAL GUIDE

(71) Applicant: Imperial Innovations Limited, London (GB)

(72) Inventors: Andrew Amis, London (GB); Ferdinando Rodriguez Y Baena, London (GB); Joshua Giles, London (GB)

(21) Appl. No.: 15/556,794

(22) PCT Filed: Mar. 10, 2016
(86) PCT No.: PCT/GB2016/050651

§ 371 Date: Sep. 8, 2017

(30) Foreign Application Priority Data
Mar. 11, 2015 (GB) 1504122.1

(51) Int. Cl. A61B 17/17; A61B 17/88; A61B 34/10

(52) U.S. Cl. CPC A61B 17/1778; A61B 2017/568; A61B 34/10; A61B 17/88

(57) ABSTRACT

A patient-specific surgical guide (10) for use in a minimally invasive procedure in a joint can include: a first contoured surface (60); and a second contoured surface (80). The first and second contoured surfaces (60, 80) are configured for engagement with respective opposing articular bone surfaces in said joint such that the guide is located in a pre-operatively determined position and orientation within the joint relative to both bones (12, 14). The provision of the two contoured surfaces for opposing bone surfaces means that the guide (10) can be securely located and retained within the joint by virtue of passive joint stiffness provided by soft tissue tension from the tissues in and surrounding the joint which is relatively unaffected by the minimally-invasive incision(s).

### FIELD OF THE INVENTION

The present invention relates generally to patient-specific surgical guides. In particular, but not exclusively, the invention relates to such guides for use in minimally invasive surgical procedures within joints, whereby the guide is located in a pre-operatively defined position and orientation within the joint relative to both articular bone surfaces thereof.

### BACKGROUND TO THE INVENTION

Surgical guides are used to assist surgeons during surgical procedures, for example to align bones during joint replacement procedures, and to guide tools for preparing bone surfaces to receive prosthetic components.

Patient-specific guides having a contoured surface to mate with a corresponding articular bone surface are known. The contoured surface may be determined pre-operatively, through techniques including the use of computer-assisted image methods based on three-dimensional images of the patient's anatomy reconstructed from MRI, CT, ultrasound, X-ray, or other three- or two-dimensional medical scans of the patient's anatomy, to ensure that the guide fits closely to the bone surface once located in position during a surgical procedure. Opposing joint surfaces each require their own single-surfaced guide. Such guides having a single surface require the surgeon to manually compress them against the articular surface in order to be kept in place (or alternatively to use screws, or other means, to keep it in place once they are happy with its location).

Moreover, in order to place such known guides in position, and then to use the guides, for example for placement of guide pins in the opposing bone surfaces of the joint, it is typically required for the surgeon to make a relatively large incision, or series of incisions. This is so as to be able to dislocate the joint to provide the necessary clear access and passage both for placement of the guide(s) into position and for insertion of the guide pin(s) using the guides—usually perpendicular to the associated articular surface—at each of the two bones. A perpendicular view of the articular surfaces is needed in order to insert the guide pins in position.

### SUMMARY OF THE INVENTION

According to a first aspect of the invention, defined by the accompanying claim 1, there is provided a patient-specific surgical guide for use in a minimally invasive procedure in a joint, the guide comprising: a first contoured surface; and a second contoured surface; wherein the first and second contoured surfaces are configured for engagement with respective opposing articular bone surfaces in said joint such that the guide is located in a pre-operatively determined position and orientation within the joint relative to both bones.

Such a patient-specific guide streamlines guide placement, because it is effectively auto-aligned and oriented within the joint, once inserted between the opposing bone surfaces, by virtue of the two contoured surfaces and a clamping effect produced by soft tissue tension across the joint, which is maintained because of the minimally-invasive nature of the procedure. Because both articular surfaces are mapped onto the guide, the joint can apply its natural compressive force which, together with some manipulation of the joint by the surgeon, will urge the guide to self-locate because it wants to find its lowest energy state (i.e. the joint orientation with the least force).

The required joint exposure is also minimised. The guide can be used in a minimally invasive setting where the joint is not dislocated.

In addition, the guide enables new means of bone alignment and preparation, as described below in the context of optional further features as set out in dependent claims. In particular, by having both articular surfaces on the guide, the bones can be locked relative to each other, which in turn allows guide pins to be driven into place without having to have a perpendicular view of the articular surface (because they can be driven through the adjacent bone from outside the joint). This again minimizes invasiveness and is not possible with currently available single surface guides.

In one embodiment, the guide comprises a monoblock component.

In an alternative embodiment, the guide is modular, comprising: a first modular component on which the first contoured surface is formed; a second modular component on which the second contoured surface is formed; and means for securing the first modular component to the second modular component. Alternatively, the two modular parts may be split in such a way that each has a part of both articular bone contours, and the two modular parts may be assembled to the bone ends from a transverse direction. According to such modular embodiments, the means for securing the first modular component to the second modular component may comprise respective mating features on said first and second modular components. Those respective mating features may comprise a groove on one of the first and second modular components and a corresponding tongue on the other of the first and second modular components. Alternatively, any other suitable mating, e.g. male and female, components could be used instead. The individual components may themselves be split into sub-components that can be secured together by suitable fixation means. One advantage of a modular construction is that it allows for greater intra-operative flexibility to the surgeon, because the different components or sub-components can conceivably be swapped-out for others, or removed to allow for improved access within and around the joint during surgery. Also, it is conceivable that the components and/or sub-components can be inserted through the minimally-invasive incision(s), for assembly together within the confines of the joint. Smaller components and sub-components of course require smaller incisions.

The modular guide may further comprise a spacer for insertion between the first and second modular components, wherein the spacer comprises part of said means for securing the first modular component to the second modular component. The provision of a spacer allows for the thickness of the assembled modular guide to be adjusted, which can be useful to allow for the surgeon to take into account different joint laxities, for example.

The first contoured surface may comprise a three dimensional surface closely mateable in only a single position with one of said articular surfaces. Likewise, the second contoured surface may comprise a three dimensional surface closely mateable in only a single position with the other of said articular surfaces. In conjunction, these mating surfaces help to ensure that the guide can be fitted into a unique position, which properly-located position can easily be determined by the surgeon through feel when inserting the guide in the joint.

The first and second contoured surfaces may further be configured to lock the articular bone surfaces in a pre-operatively determined configuration relative to the guide and hence relative to one another. As such, the bones may be set in a particular clinically useful pose.

The guide may further comprise at least one datum, so as to provide a fixed point of reference for the surgeon.

The guide may further comprise means for attachment of and/or guiding of at least one surgical instrument or intra-operative tracking marker or sensor. The guide according to this embodiment may further comprise an additional modular component for attachment to said guide in a pre-operatively determined position and orientation relative to the guide, for guiding at least a selected one of the following exemplary surgical procedures: guide wire insertion, drilling, cutting, reaming, resecting, augmenting, injecting, imaging and screwing. When for use in guide wire insertion, the guide may further comprise a clearance hole and an associated slot such that a guide wire can be inserted through the hole for attachment to at least one of the bones and such that the guide is removable from the joint with the wire in place by the passage of the wire through the slot.

The guide may further comprise at least one fixation hole for receiving a fixing screw or other attachment means to secure the guide to the joint, in use. The guide may also or instead further comprise at least one hole for use in making a reference mark on the underlying bone surface, which mark can be used to help guide subsequent surgical steps. In certain embodiments, the fixation hole may be used for making that reference marking, for example by virtue of the hole left by the screw acting as marker.

The guide may be manufactured from a medical-grade polymer or metallic material including but not limited to: polyethylene, 316 steel, nylon 6, acrylic, cobalt chrome, titanium and PEEK. These materials are especially conducive to the guide being manufactured using additive manufacturing, which is a particularly good way to make guides specific to each patient and procedure with minimal wastage of stock material.

The guide may be for a shoulder joint, in which case the first contoured surface is a humerus-contacting surface, sized and shaped to substantially match the geometry of at least a portion of the patient's humerus, and in which the second contoured surface is a glenoid-contacting surface, sized and shaped to substantially match the geometry of at least a portion of the patient's glenoid cavity. In alternative embodiments, the guide may instead be for other articular joints, such as the knee, hip or elbow. In further alternative embodiments, the guide may be adapted for use in association with other opposing bones or bone portions, such as for use in realignment of bone fractures, or in sarcoma surgery.

The guide may further comprise chamfered surfaces positioned to aid in separation of the two bone surfaces, against the passive joint tension, during insertion of the guide.

The guide may be at least partially coated with a low friction coating, so as to assist in insertion of the guide into between the opposing bone surfaces.

Where the guide is modular, the first and second modular components may include respective means for connection to associated first and second arms of a pivoting surgical instrument and the means for securing the first modular component to the second modular component may comprise a mechanism for locking the first and second arms relative to one another. By way of example, the first and second modular components may each include a hole for securely receiving a prong on the end of the respective arms of the surgical instrument, and the locking mechanism may be a ratchet, as known in self-retaining soft tissue retractor instruments.

### BRIEF DESCRIPTION OF DRAWINGS

Embodiments of the present invention will now be described, by way of example only, with reference to the accompanying drawings, in which:

FIG. 1 shows an posterior-inferior isometric view of an assembled modular guide according to one embodiment, located in situ within a shoulder joint, with portions of the humerus and scapula shown in wire-frame;

FIG. 2 is a close-up view of the guide of FIG. 1;

FIG. 3 corresponds to FIG. 2, but from a posterior viewpoint;

FIG. 4a corresponds to FIGS. 2 and 3, but from an anterior-inferior isometric viewpoint;

FIG. 4b corresponds to FIG. 2, but with the guide removed to show the underlying bone surfaces;

FIGS. 5a-5c depict the assembled modular guide of the preceding figures, in respective inferior, anterior-inferior isometric and posterior views, but with the bones removed for clarity—note that FIG. 5b corresponds to FIG. 4 and that FIG. 5c corresponds to FIG. 3;

FIGS. 6a and 6b depict a humeral-side component of the guide in respective anterior and posterior views;

FIGS. 7a and 7b depict the humeral-side component of FIGS. 6a and 6b, but with a modular tab sub-component removed;

FIGS. 8a-8d depict the modular tab sub-component of the humeral-side component in respective anterior, posterior, anterior-superior and superior views;

FIGS. 9a-9d depict a scapular-side component of the guide in respective anterior, anterior-inferior, anterior-inferior-lateral and posterior-superior isometric views;

FIG. 10 is a posterior view of an optional spacer for insertion between the humeral- and scapula-side components of the guide; and

FIGS. 11 a-d depict an embodiment in which the humeral-side component includes a slot feature for the passage of a guide wire therethrough, as well as drilling steps for its insertion.

### DETAILED DESCRIPTION

The following description will be made in the context of a patient-specific surgical guide for use in a minimally invasive surgical procedure on a patient's shoulder joint. It should be understood, however, that the principles and teachings can be applied mutatis mutandis to produce guides useable in other articular joints, such as the knee, hip or elbow. Suitable applications include, but are not limited to, total or partial joint replacement procedures and soft-tissue repairs such as ligament repair or replacement.

The term ‘minimally invasive surgical procedure’ is considered to have an established meaning within the art and is intended to encompass, inter alia, arthroscopic and mini-open surgical approaches, including those using multiple minimally-invasive incisions.

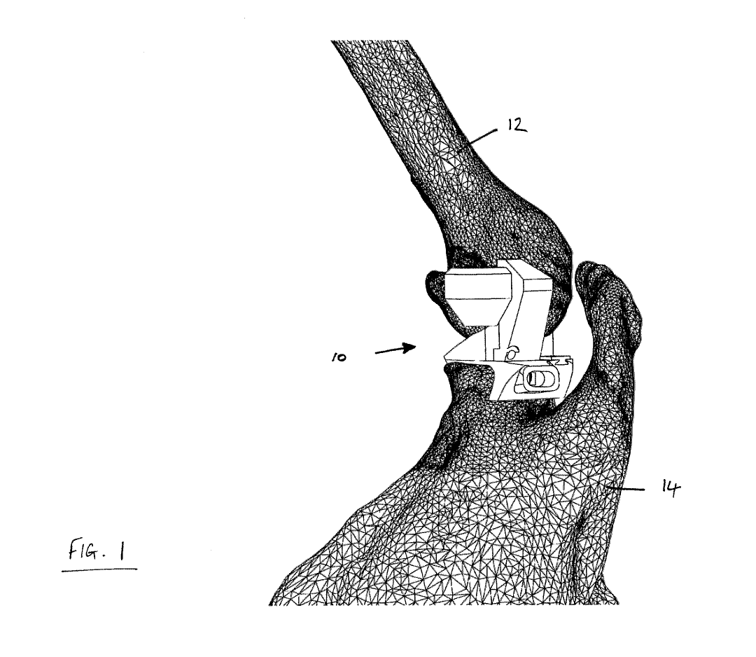

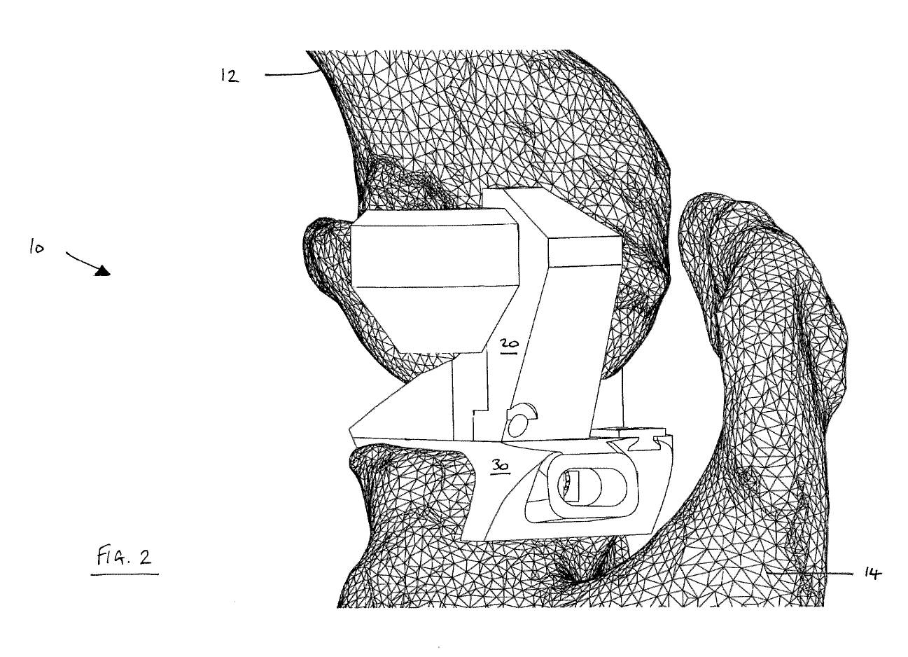

A patient-specific surgical guide assembly (‘PSG’) 10 is shown located in situ between a patient's humerus 12 and scapula 14 in FIGS. 1 to 4. The PSG 10 is modular, comprising a first modular component 20 for location at the humeral side of the joint (the humeral-side component 20) and a second modular component 30 for location at the scapula side of the joint (the scapula-side component 30).

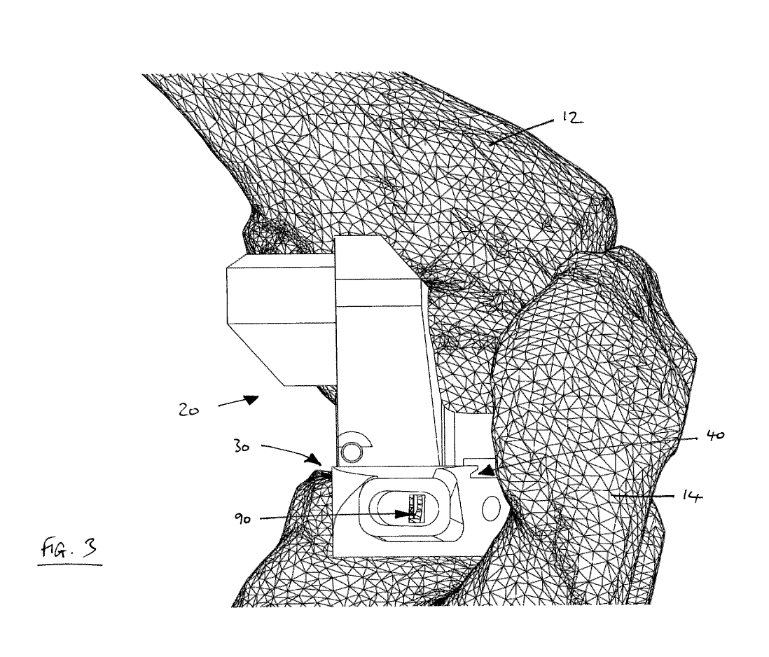

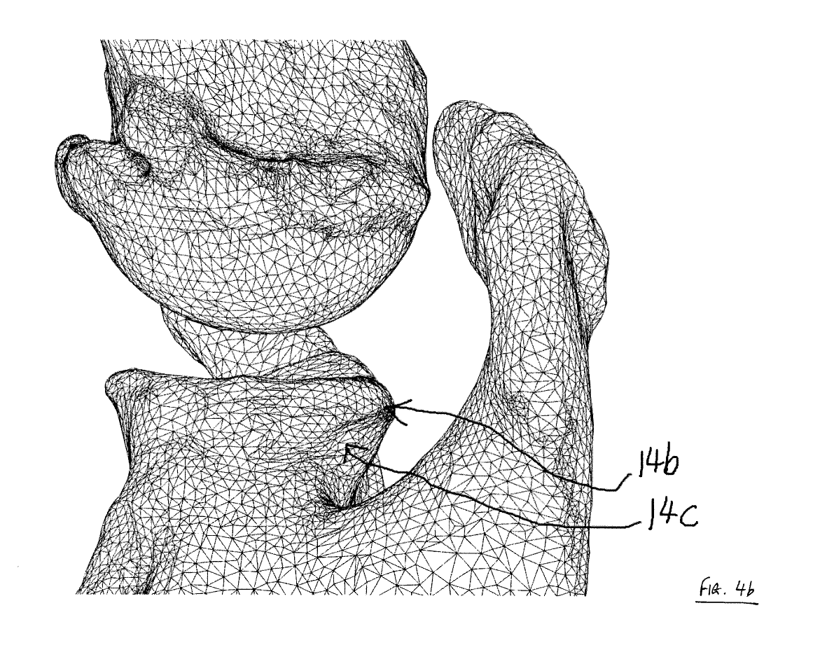

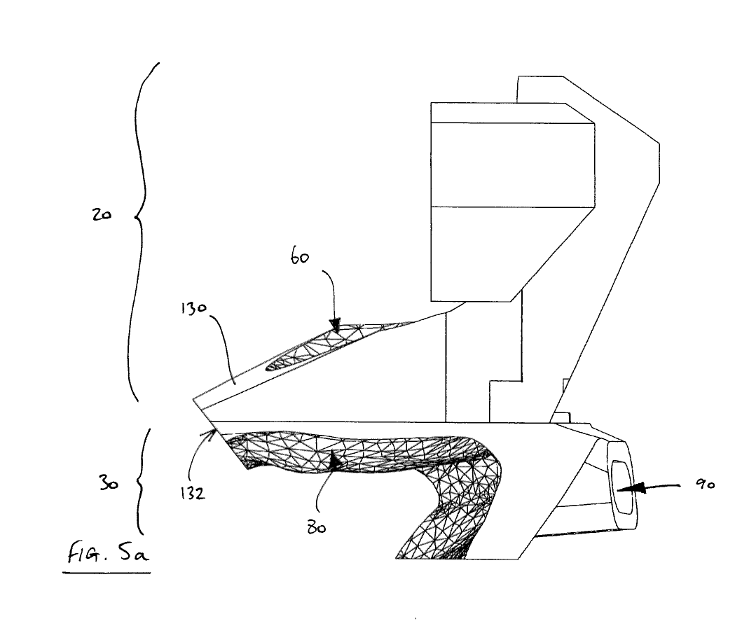

The humeral-side component 20 and the scapula-side component 30 are secured to one another via a mating connection 40, perhaps best seen in FIGS. 3 and 5c. The mating connection 40 here comprises a tongue 42 protruding from an end of the humeral-side component 20 closest to the scapula-side component 30, said tongue 42 slidingly received in a mating groove 44 formed on an opposing end of the scapula-side component 30. It will be understood that numerous alternative mating connections are envisaged and that the skilled person would be able to conceive of suitable such connections. By way of example only, the tongue 42 could project from the scapula-side component 30 with the corresponding groove 44 being in the humeral-side component 20 or, rather than a sliding tongue and groove arrangement, the secure connection could be made via a series of interconnecting fasteners.

As best seen in FIG. 5b, the humeral-side component 20 has a contoured surface 60, customised to match corresponding contours on the surface of the patient's humerus 12. This first contoured surface 60 will be described in greater detail below. Likewise, the scapula-side component 30 has a contoured surface 80, customised to match corresponding contours on the surface of the patient's scapula 14, and more particularly the glenoid and surrounding surfaces. This second contoured surface 80 will also be described in greater detail below.

The first and second contoured surfaces are determined pre-operatively, through known techniques including the use of computer-assisted image methods based on three-dimensional images of the patient's joint anatomy reconstructed from MRI, CT, ultrasound, X-ray, or other three- or two-dimensional medical scans of the patient's anatomy.

In the pre-operative planning stage, imaging data of the relevant anatomy of a patient can be obtained using one of medical imaging methods described above, as needed for joint modeling, mechanical/alignment axis determination or for other alignment purposes. The imaging data obtained and other associated information can be used to construct a three-dimensional computer image of the joint or other portion of the anatomy of the patient. An initial pre-operative plan can be prepared for the patient in image space and can include bone or joint preparation, planning for resections, milling, reaming, broaching, implant selection and fitting, as well as designing patient-specific guides, templates, tools and alignment methods for the surgical procedure.

The imaging data can thus be used to design the first and second contoured surfaces 60, 80 of the PSG 10 to ensure that when the PSG 10 is located between the opposing articular bone surfaces 12, 14 in the patient's joint during the surgical procedure, several objectives are achieved.

A first objective is to ensure that the PSG is located in a pre-operatively determined position and orientation within the joint relative to both bones. This is known as registration of the guide to the joint, and assists the surgeon in knowing that the guide is properly located before continuing further with the procedure so that the PSG 10 can be used as a platform for further surgical steps requiring knowledge of a bone's position and orientation. A PSG 10 according to the invention is capable of achieving automatic self-location to the desired position within the joint through a combination of said first and second contoured surfaces 60, 80 and the passive tension of surrounding trans-articular soft tissues (e.g. muscles, tendons, ligaments, and/or joint capsule) which will tend to result in the bones 12, 14 of the joint positioning and orienting themselves in the desired configuration, which is that with the lowest passive tension allowable when the PSG 10 is present within the joint.

To this end, it is preferable for at least one (and more preferably both) of the first and second contoured surfaces 60, 80 to comprise a three-dimensional surface that is closely mateable with the associated bone surface in only a single position.

A second objective is to ensure that the bones are ‘locked’—i.e. located in a fixed, pre-operatively determined configuration—relative to the PSG and hence to each other. This is known as alignment. The PSG 10 according to this embodiment would thus allow new bone preparation techniques to be performed which simultaneously prepare both bones 12, 14 given that the relative configuration of the two bones is known and can be pre-operatively chosen to correspond to a clinically useful pose.

By way of example, a relatively shallow or small contoured surface might contain enough ‘landmarks’ i.e. unique points and regions matching those of an associated bone surface such that the surface can only be fitted neatly to the bone in a single, unique position. However, the bone could be susceptible to being shifted out of that position, for example by relative translational movement of the bone away from the surface, or through a relative twisting action.

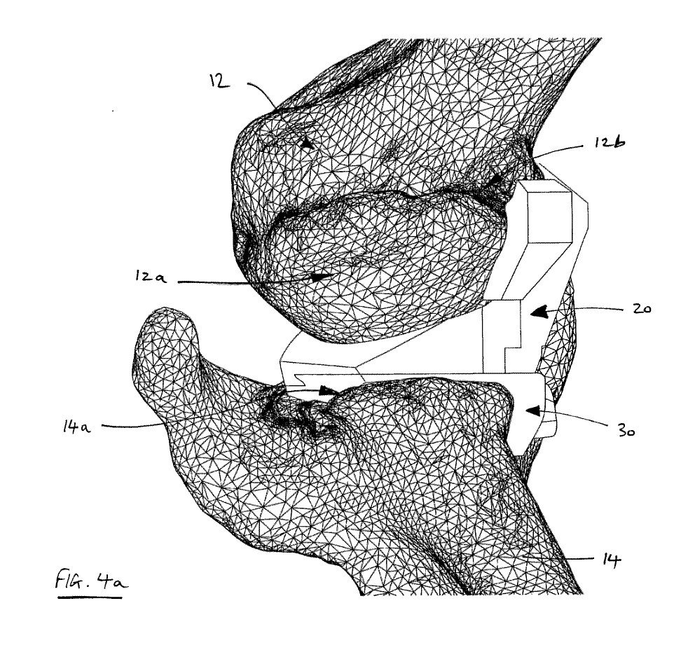

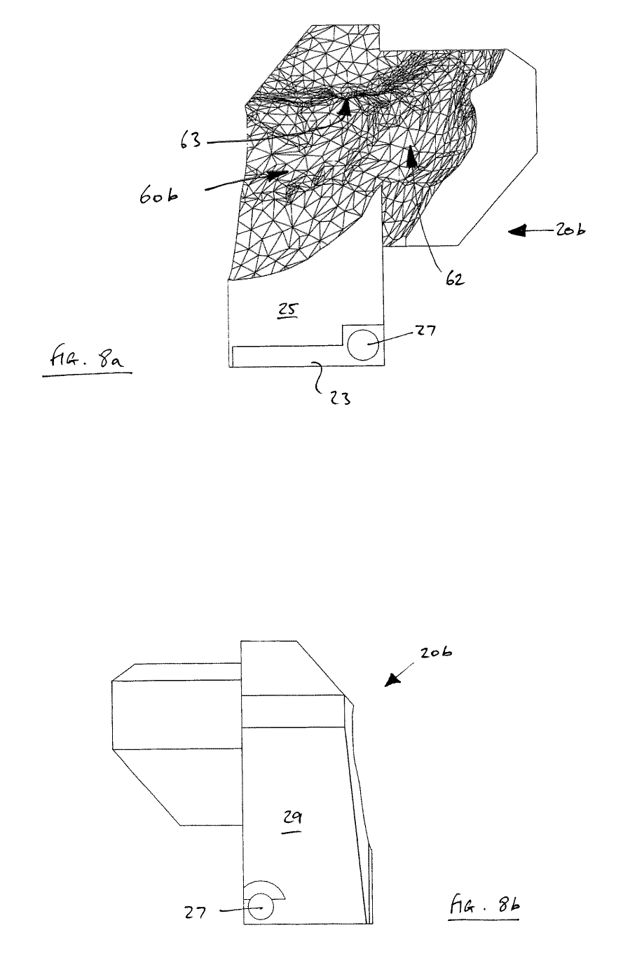

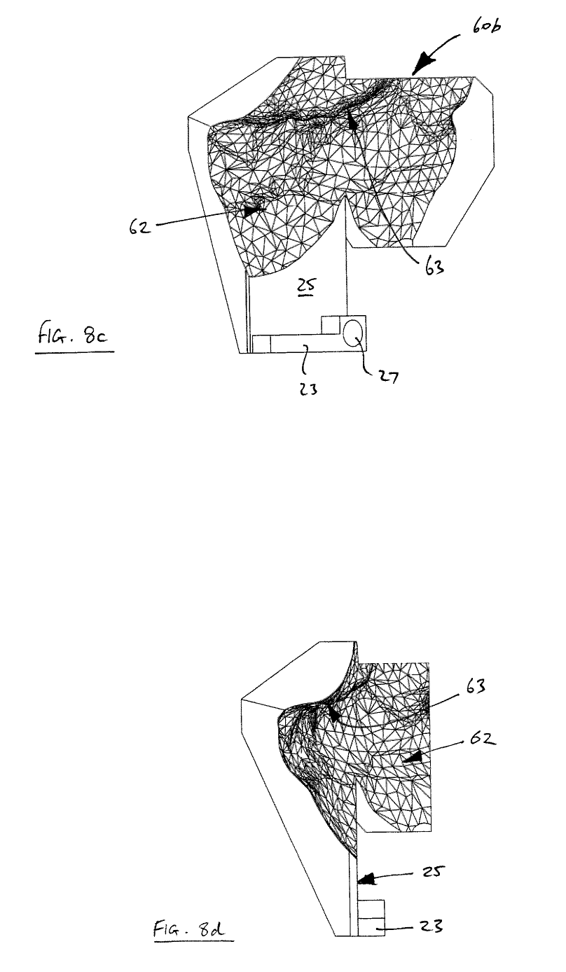

To mitigate against that, the contoured surface may be extended, for example to ‘hook’ over a portion of the bone. In the exemplary example described herein, the contoured surface 60 of the humeral-side component 20 comprises a relatively shallow, generally concave portion 61 contoured to match the convex contours of at least part of the end of the patient's humeral head 12a. The contoured surface 60 further comprises a sidewall extension portion 62 that enshrouds at least part of the side of the patient's humeral head 12a, and an overhanging lip 63 that fits under at least a part of the patient's humeral head 12a, within the humeral neck 12b. In this way, the contoured surface 60 not only accommodates the humerus 12 in a single position relative to the PSG 10, but also resists relative movement (translation and/or rotations) between the humerus 12 and the PSG 10 once located in position. FIGS. 4a, 5b and 6a, as well as FIGS. 8a-8d perhaps best show these features.

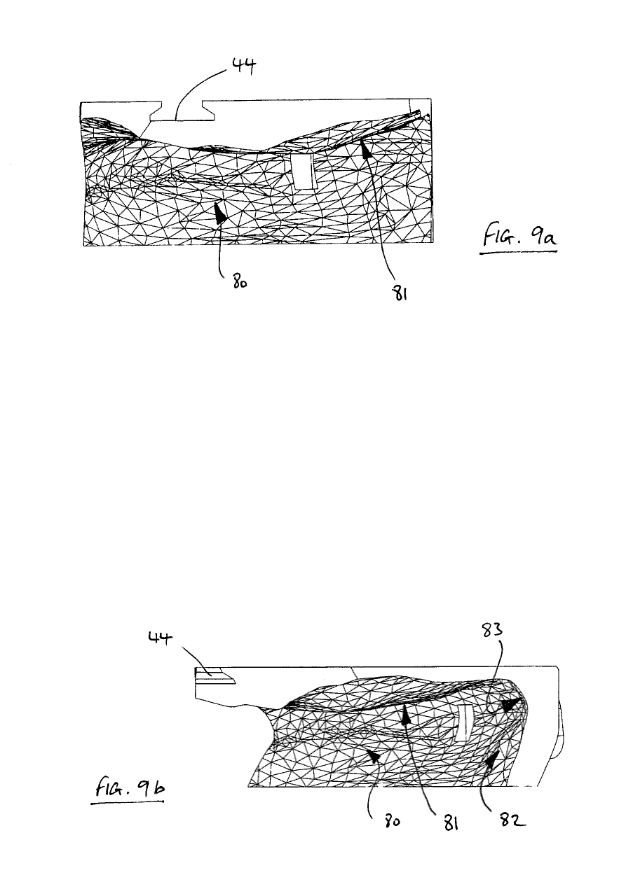

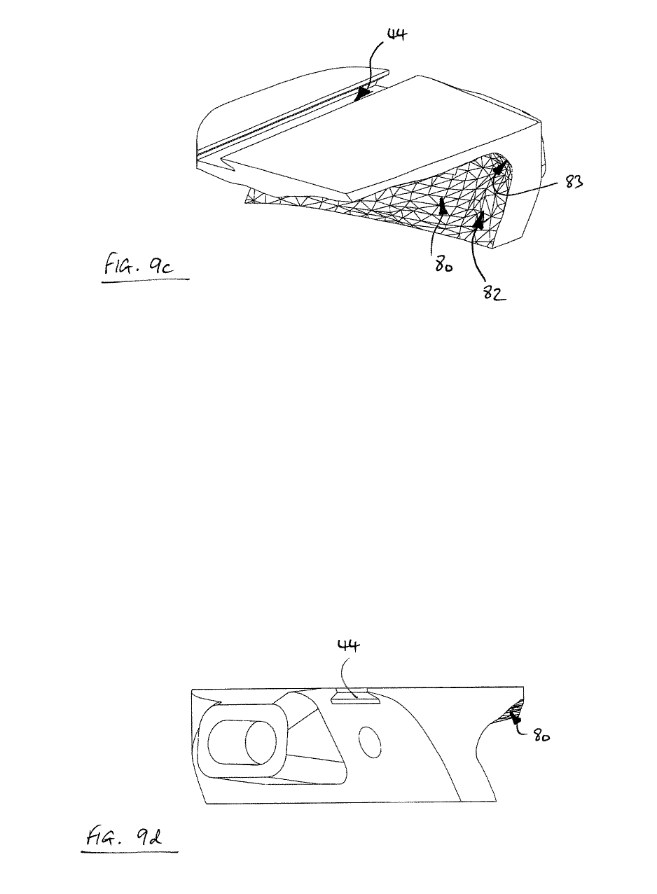

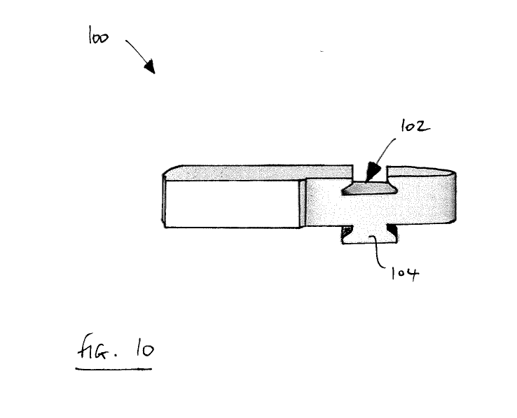

The contoured surface 80 of the scapula-side component 30 comprises a relatively shallow, generally convex portion 81 contoured to match the concave contours of at least part of the end of the patient's glenoid cavity 14a. The contoured surface 80 further comprises an inwardly-angled sidewall extension portion 82 that enshrouds and accommodates at least part of the patient's posterior glenoid rim 14b, and posterior glenoid vault 14c. In this way, the contoured surface 80 not only accommodates the scapula 14 in a single position relative to the PSG 10—particularly by accommodating the patient's posterior glenoid rim 14b within a region 83 formed between the convex portion 81 and the sidewall extension portion 82, but also resists relative movement (translation and/or rotations) between the scapula 14 and the PSG 10 once located in position. FIGS. 4a, 4b, 5b and 9a-9c perhaps best show these features.

In embodiments in which the surface contours are extended to lock the PSG 10 in position relative to the bones 12, 14 and vice versa, the very features that resist the relative movement (e.g. the sidewall 62 and lip 63 of the contoured surface 60, or the sidewall 82 of the contoured surface 80) also make it more difficult to fit the PSG in place. So, one or both of the modular components 20, 30 can be split into sub-components to help in fitting their respective contoured surfaces 60, 80 to and around the associated bone surfaces.

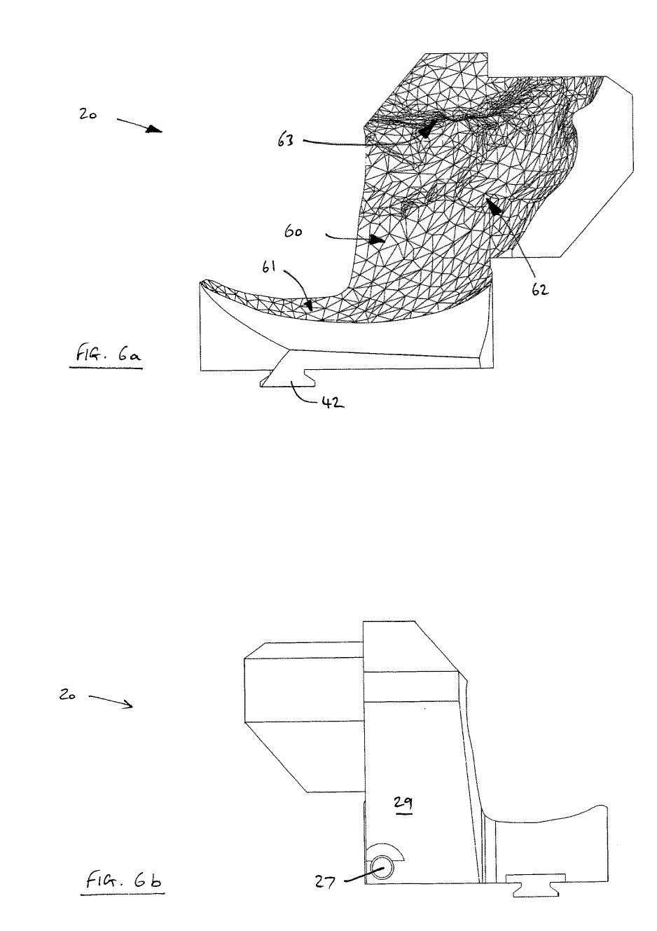

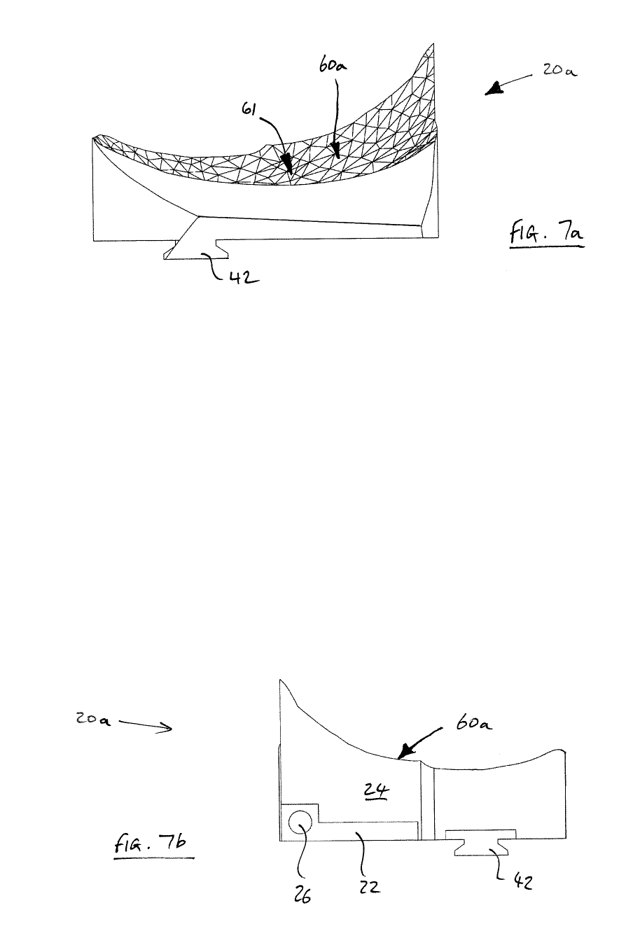

In the exemplary embodiment described herein, the humeral-side component 20 comprises two sub-components: a base portion 20a (see FIGS. 7a, 7b), which includes the tongue 42 that secures the humeral-side component 20 to the scapula-side component 30, and which includes a first part 60a of the first contoured surface 60; and a fin or tab portion 20b (see FIGS. 8a to 8d), which includes a second part 60b of the first contoured surface 60. Broadly speaking, the first part 60a of the first contoured surface 60 comprises the relatively shallow, generally concave portion 61, and the second part 60b of the first contoured surface 60 comprises the sidewall extension portion 62 and overhanging lip 63. However, it will be appreciated that the division between the concave portion 61 and the sidewall extension portion 62 may not be a well-defined boundary, such that the second part 60b may include part of the concave portion 61 and/or the first part 60a may include part of the sidewall extension portion 62.

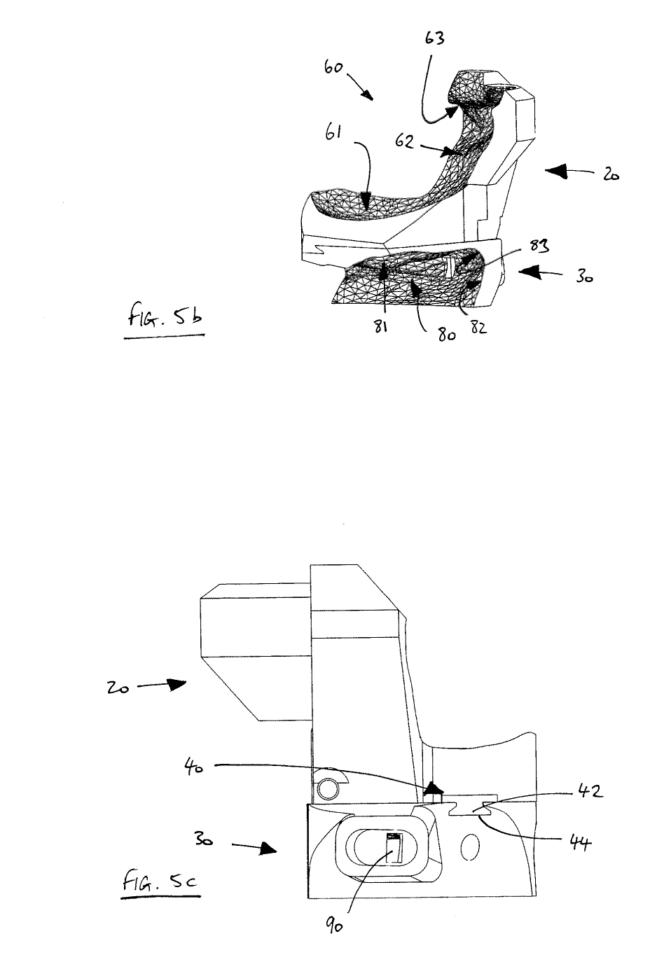

The base portion 20a includes an L-shaped recess 22 on its posterior external surface 24 adjacent the end that is closest to the scapula-side component 30. A hole 26 is formed through a widest part of the recess 22. The tab portion 20b includes an L-shaped protrusion 23 on its anterior external surface 25 adjacent the end that is closest to the scapula-side component 30, the protrusion 23 matching the recess 22. A through-hole 27 extends through the widest part of the protrusion 23 and through the rest of the adjacent part of the tab portion 20b to the posterior external surface 29. The base portion 20a and the tab portion 20b are assembled together by fitting the protrusion 23 into the recess 22, and secured together in that position by means of a fastener, such as a bolt (not shown) threaded into the hole 27. In this assembled configuration, the first and second parts 60a, 60b of the first contoured surface 60 are contiguous (see FIG. 6a).

The humeral-side component 20 may be divided into more than two sub-components if necessary and according to the patient's specific anatomy and needs, as well as the procedure being carried out with the use of the PSG. It may be beneficial to remove a sub-component during the surgical procedure, for example to provide better access to a part of the joint to allow surgical steps to be performed. An alternative embodiment may allow both the base and tab portions to be made as one piece, joined by a thin hinge line, which can be bent into the desired shape when placed within the joint, and secured in that configuration, for example by a snap-together mechanism.

As illustrated, the scapula-side component 30 is a unitary part, and it can be fitted in place by a combination of a translation and a rotation, to accommodate the patient's posterior glenoid rim 14b within the region 83 formed between the convex portion 81 and the sidewall extension portion 82. It will be understood, however, that in certain circumstances, such as those described with reference to the humeral-side component 20, it may be better to split the component into two or more sub-components, which could be secured together in the manner described above with reference to the humeral-side component 20.

The PSG 10 may include at least one datum (not shown) for use as a reference point or reference plane, for example. The PSG 10 may also or instead include means (not shown) for attachment of and/or guiding of at least one surgical instrument or intra-operative tracking marker or sensor.

Additional modular components (not shown) may be included for attachment to the PSG 10 in a pre-operatively determined position and orientation relative to the guide, for guiding at least a selected one of the following exemplary surgical procedures: guide wire insertion, drilling, cutting, reaming, resecting, augmenting, injecting, imaging and screwing. To this end, the, or each, additional modular component would either be of a pre-operatively planned patient-specific design, or would be of a standard design but with patient-specific attachment locations on the PSG 10 so as to ensure that the surgical steps are carried out in the appropriate location and orientation for that particular patient.

The PSG 10 includes a hole 90 for use in marking the underlying glenoid surface with a reference mark for subsequent steps of the procedure once the PSG has been removed. The PSG 10 may further include one or more fixation holes (not shown) for receiving a fixing screw to secure the guide to the joint, in use. In one embodiment, the hole 90 may be used as such a fixation hole, with the screw-hole made in the bone surface during placement of the screw therein acting as the reference mark. As illustrated in the exemplary embodiment, the hole 90 is provided through the scapula-side component 30, for fixation of a screw into the scapula 14—in particular the posterior glenoid rim 14b thereof, but it will be appreciated that additional or alternative hole locations could be used instead, depending for example on the patient's joint geometry and operational needs.

The PSG 10 is typically disposable and made of lightweight materials, including certain metals and polymers including, but not limited to: polyethylene, nylon 6, acrylic, PEEK, 316 steel, cobalt chrome, and titanium, as well as alloys thereof. Where made of modular components, the components could be manufactured from different materials, to take advantage of different material properties, if desirable. The PSG 10 may conveniently be manufactured using additive manufacturing techniques

In use, a surgeon would insert the components and sub-components of the PSG 10 through one or more minimally-invasive incisions for assembly within the area of the joint. Insertion of the PSG 10 into place between the bone surfaces during such a minimally-invasive procedure may require quite some force, by virtue of the fact that the soft tissues in and surrounding the joint (e.g. the joint capsule, and rotator cuffs comprised of the infraspinatus, teres minor, subscapularis, and supraspinatus) are not greatly affected by the minimally-invasive incisions, and thus continue to produce their natural and ever present tonic forces—which regularly compress the two articular surfaces together and prevent the joint from dislocating—throughout the procedure. Just as it helps to hold the natural components of the joint firmly in place, the passive joint stiffness provided by the soft tissue tension helps to retain the PSG 10 firmly in place once inserted, thereby ensuring a stable guide location that the surgeon can rely on being accurately located.

To assist in the insertion of the PSG 10 into location between the opposing articular bone surfaces and against the latent soft tissue tension, the PSG 10 may include one or more tapered or chamfered surfaces so that it has a general wedge shape, which would help to separate the bone surfaces. In the illustrated embodiment, such tapered surfaces are provided on an anterior side of the PSG: the humeral-side component 20 including a first tapered surface 130; and the scapula-side component 30 including a second tapered surface 132, a portion of which in fact continues across on to the humeral-side component. This is best seen in FIGS. 5a and 11. The location of the tapered surfaces 130, 132 on the anterior side of the PSG 10 will assist in its insertion from the posterior.

Specifically, humeral-side base portion sub-component 20a and scapula-side component 30 will typically be assembled together outside of the patient using the above described fixation method, while the humeral-side tab sub-component 20b will not be attached until after the above components with their tapered shape are inserted into between the bone surfaces. Once assembled together, these combined components 20a, 30 are inserted typically through a posterior incision, while sub-component 20b will be inserted within the soft tissue envelope of the joint, slotted onto component 20a by mating surfaces 22 and 23 and connecting them using a fastener. Once connected, the scapula-side component 30 can be confirmed to be properly located as a result of surface 80 mating with the scapula 14. Subsequently, the humerus 12 can be passively adjusted by the surgeon until it can be felt to be properly located relative to surface 60 (e.g. the humerus feels physically locked in place due to the rigid connection the guide creates between the scapula and humerus).

To further assist in insertion, one or more of the surfaces of the PSG 10 may be at least partially coated with a low-friction coating.

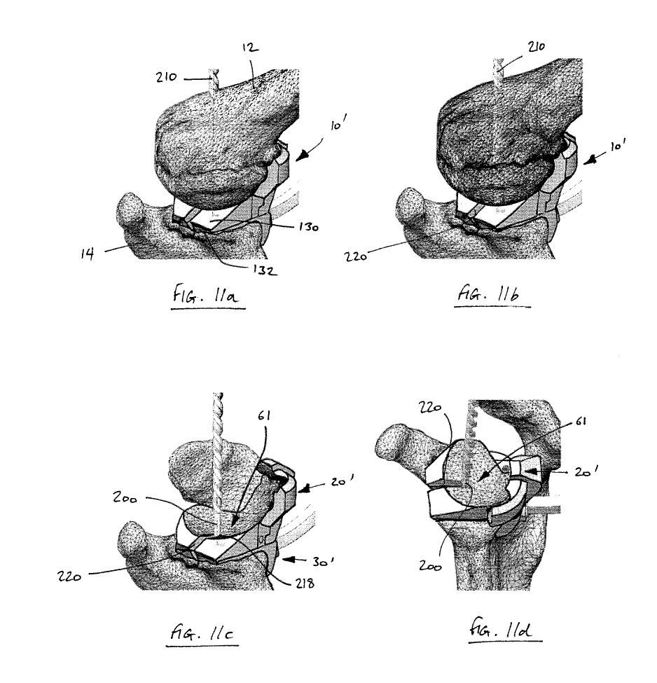

Because it can be difficult to determine pre-operatively how much tension the joint will be under from the soft tissues, it is desirable to allow for the ‘thickness’ of the PSG 10 to be altered intra-operatively so that the resulting soft tissue tension with the PSG 10 in place can be altered accordingly. To this end, a spacer 100, as shown in FIG. 10, may be provided to be interposed between the humeral-side component 20 and the scapula-side component 30. The spacer 100 includes a groove 102 that corresponds to the groove 44 in the scapula-side component 30 and which is therefore able to slidingly receive the tongue 42 of the humeral-side component 20 for secure connection thereto. The spacer 100 further comprises, on an opposing surface, a tongue 104 that corresponds to the tongue 42 of the humeral-side component 20 and which is therefore able to be slidingly received within the groove 44 on the scapula-side component 30 for secure connection thereto. In embodiments including such a spacer 100, the spacer could be fitted between the humeral-side sub-component 20a and scapula-side component 30 outside of the patient, and the assembly 20a, 100, 30 would be inserted typically through a posterior incision as described above, with the tab sub-component 20b being secured to the assembly once the assembly is located between the humerus 12 and the scapula 14. Alternatively, the humeral facing and scapular facing components may be inserted into the joint, and the spacer part then inserted between them, so as to tense the surrounding joint soft tissues.

A range of spacers 100 of different thicknesses may be provided to allow even greater flexibility to the surgeon in selecting the ‘thickness’ of the PSG 10 intra-operatively, so that an optimum fit and optimum passive joint stiffness can be selected. More than a single spacer 100 could be used; a number of spacers being stacked for insertion between the humeral-side sub-component 20a and the scapula-side component 30.

Note that the use of zero or more spacers 100 will depend on the laxity of the patient's joint and the surgeon's experience/preference, whereby spacers can be added to increase the passive tension in a lax joint, and thus produce a greater compressive force on the inserted components to help in properly maintaining their position.

In one embodiment, rather than comprising an assembly of component parts 20, 30, the PSG 10 may instead comprise a single, monoblock component (not shown), typically formed from a single piece of material. An advantage of a monoblock unitary construction of the PSG is that it likely to be stiffer and stronger than a similar PSG of modular construction. It would also not require assembly within the confines of the joint during the surgical procedure, so could be less fiddly to insert. However, the larger overall dimensions of the monoblock PSG as compared to the modular components of an equivalent modular PSG would likely require a larger surgical incision and might therefore not be suitable for minimally-invasive procedures. It is also not possible to insert a spacer component to adjust the ‘thickness’ of the PSG to find the tightest possible fit between the bones. To mitigate for this fact, a range of monoblock PSGs of different ‘thicknesses’ could be provided, so that the surgeon can select the optimum size intra-operatively.

In an alternative embodiment, the two bone-facing components may be adapted for attachment to a surgical instrument configured in the same way as a self-retaining soft tissue retractor instrument with a central pivot between two parts such that it works like a scissors mechanism. For example, the bone-facing components may have holes in them which mate with prongs on the ends of the arms of the retractor instrument and which also have means to ensure that they lock into a predetermined position. Thus, in use, the two bone-facing components may be inserted into the joint while close together (back to back), and then the surgeon may squeeze the handles of the retractor together, thereby tensing the surrounding soft tissues of the joint as the bone-facing components move apart and are retained in the spaced-apart position by means of a ratchet mechanism in the retractor instrument.

As known in the art, certain procedures require the insertion of a guide wire 200, and an associated drilling step. This is illustrated in FIGS. 11 a-d. FIGS. 11a and b show the passage of a driver 210 through the patient's humerus 12 and beyond, through the PSG 10′, into the scapula 14, the bones being held secure in position by the PSG 10′, and the driver 210 being aligned by reference to the PSG 10′, optionally through use of an additional guide component (not shown) to aid in accurately inserting the guide wire 200 extra-articularly. In FIGS. 11c and d, the humerus is removed for clarity and the passage of the driver 210 and guide wire 200 can be seen through the generally concave portion 61 of the contoured surface 60, which includes a clearance hole 218 positioned in a pre-operatively planned location and orientation for proper positioning of the guide wire during the procedure. In particular, the relative configuration of the two primary contoured surfaces 60, 80 as dictated by the pre-operative plan ensures that the desired axes of the guide wire 200 for each bone 12, 14 are collinear, thus allowing a guide wire 200 to be driven along this axis (typically using said attached secondary guide) to concurrently produce the required guide for both bones.

In particular, the guide wire 200 according to this embodiment is relatively short, for insertion into just the glenoid, not extending back into the humerus. This short guide wire portion 200 is attached to a mating driver 210 to drive the wire through the humerus 12 and into the glenoid. As known in the art of guide wire placement, the distal end of the guide wire includes features (not shown) that enable it, as driven by the attached bit 210, to ‘self-drill’ through bone. The short guide wire 200 is thus able to be driven through the humerus and into the glenoid without the need for a pilot hole. Once located in position within the glenoid, the guide wire 200 can be detached from the driver 210, acting in effect as a pin in the glenoid, aligned to the pre-determined, properly-oriented axis along which it was inserted, for use to guide further steps in the procedure. The driver 210 would then be retracted back through the humerus, leaving a bone tunnel. The bone tunnel produced through the humerus 12 by the passage of the short guide wire 200 and associated driver 210 can itself act as a humeral guide. In traditional surgery, an independent guide wire would be inserted into the humerus, being inserted from the articular surface and oriented as required, but in this embodiment, the humeral guide wire can be replaced by the bone tunnel because the bone tunnel and the guide wire 200 are necessarily aligned along a common axis as a result of the insertion technique. This can be advantageous because the shorter length of the guide wire 200 means that the technique can be undertaken in a more confined space while still providing a means of guiding surgical steps (guide pin for the scapula, and bone tunnel for the humerus).

The driver 210 may be mated to the guide wire 200 by any suitable connection that allows the guide wire 200 to be driven with sufficient force for insertion through the humerus 12 and into the glenoid. Suitable examples would include, but are not limited to, screwdriver- or allen-type bits and associated mating slots.

In the illustrated embodiment, the driver comprises a drill bit 210 with a connection feature (not shown) incorporated into its distal end. The use of a drill bit 210 as the driver allows for a relatively wide diameter bone tunnel to be formed through the humerus 12 simultaneously with the driving of a relatively narrow diameter short guide wire 200 into the glenoid. Alternatively, the bone tunnel could be enlarged using a conventional drill bit in a separate and subsequent step to the insertion of the guide wire 200.

A through slot 220 is provided from the clearance hole 218 to the anterior side of the PSG 10′ so that the PSG 10′ can be removed from the joint once the guide wire 200 has been fixed in place, the guide wire 200 ‘passing through’ the slot 220 as the PSG 10′ is removed to the posterior.

In certain embodiments, a further degree of freedom could be provided in the PSG, for example by including features on the mating surfaces of adjacent components or sub-components that allow them to rotate relative to one another before they are locked or fixed in position. This would provide the surgeon with additional flexibility with regards to limb orientation. In other words, one could pre-plan a range of valid relative bone configurations, then design and manufacture a PSG incorporating features that allow the surgeon the scope to select any one of those configurations once the patient is prepped and on the operating table.

In addition to the above-described applications in articular joint structures, similar principles can be used to produce patient-specific guides for use in realignment of bone fractures, or in sarcoma surgery. In this context, rather than the guide being made to fit between two opposing articular joint surfaces, it would instead fit in between two bone fragments where part of the original bone has been lost, for example either due to the compressive nature of the fracture or where it has been removed deliberately in a surgical procedure. The two bone fragments can be properly spaced and aligned by the guide, then plated.

It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail. Accordingly, individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure as defined by the accompanying claims.

1. A patient-specific surgical guide for use in a minimally invasive procedure in a joint, the guide comprising:
a first contoured surface; and
a second contoured surface;
wherein the first and second contoured surfaces are configured for engagement with respective opposing articular bone surfaces in said joint such that the guide is located in a pre-operatively determined position and orientation within the joint relative to both bones.

2. The guide of claim 1, wherein the guide comprises a monoblock component.

3. The guide of claim 1, wherein the guide is modular, comprising:
a first modular component on which the first contoured surface is formed;
a second modular component on which the second contoured surface is formed; and
means for securing the first modular component to the second modular component.

4. The guide of claim 3, wherein the means for securing the first modular component to the second modular component comprise respective mating features on said first and second modular components.

5. The guide of claim 4, wherein the respective mating features comprise a groove on one of the first and second modular components and a corresponding tongue on the other of the first and second modular components.

6. The guide of an of claim 3, further comprising a spacer for insertion between the first and second modular components, wherein the spacer comprises part of said means for securing the first modular component to the second modular component.

7. The guide of claim 1, wherein the first contoured surface comprises a three dimensional surface closely mateable in only a single position with one of said articular surfaces.

8. The guide of claim 1, wherein the second contoured surface comprises a three dimensional surface closely mateable in only a single position with one of said articular surfaces.

9. The guide of claim 1, wherein the first and second contoured surfaces are further configured to lock the articular bone surfaces in a pre-operatively determined configuration relative to the guide and hence relative to one another.

10. The guide of claim 1, further comprising at least one datum.

11. The guide of claim 1, further comprising means for attachment of and/or guiding of at least one surgical instrument or intra-operative tracking marker or sensor.

12. The guide of claim 11, further comprising an additional modular component for attachment to said guide in a pre-operatively determined position and orientation relative to the guide, for guiding at least a selected one of the following exemplary surgical procedures: guide wire insertion, drilling, cutting, reaming, resecting, augmenting, injecting, imaging and screwing.

13. The guide of claim 12, when for use in guide wire insertion, the guide further comprising a clearance hole and an associated slot such that a guide wire can be inserted through the hole for attachment to at least one of the bones and such that the guide is removable from the joint with the wire in place by the passage of the wire through the slot.

14. The guide of claim 12, further comprising at least one fixation hole for receiving a fixing screw to secure the guide to the joint, in use.

15. The guide of claim 1, manufactured from a polymer or metallic material including but not limited to: polyethylene, 316 steel, nylon 6, acrylic, cobalt chrome, titanium and PEEK.

16. The guide of claim 15, manufactured using additive manufacturing.

17. The guide of claim 1, wherein the guide is for a shoulder joint, wherein the first contoured surface is a humerus-contacting surface, sized and shaped to substantially match the geometry of at least a portion of the patient's humerus, and wherein the second contoured surface is a glenoid-contacting surface, sized and shaped to substantially match the geometry of at least a portion of the patient's glenoid cavity.

18. The guide of claim 1, further comprising chamfered surfaces positioned to aid in separation of the two bone surfaces during insertion of the guide.

19. The guide of claim 1, wherein the guide is at least partially coated with a low friction coating.

20. The guide of claim 3, wherein the first and second modular components include respective means for connection to associated first and second arms of a pivoting surgical instrument and wherein the means for securing the first modular component to the second modular component comprises a mechanism for locking the first and second arms relative to one another.
